(19) United States
(12) Patent Application Publication
(10) Pub. No.: US 2016/0136095 A1
(43) Pub. Date: May 19, 2016

(54) METHODS FOR TREATING EPILEPSY OR SEIZURE DISORDERS

(71) Applicant: PixarBio Corporation, Medford, MA (US)

(72) Inventors: JASON M. CRISCIONE, CHELMSFORD, MA (US); NICHOLAS B. WERTH, READING, MA (US); ROBERT S. LANGER, NEWTON, MA (US); FRANCIS M. REYNOLDS, SALEM, NH (US); HAINING DAI, ROCKVILLE, MD (US); PATRICK A. ARMSTRONG, BOSTON, MA (US); XI CHEN, ACTON, MA (US)

(21) Appl. No.: 14/943,846

(22) Filed: Nov. 17, 2015

Related U.S. Application Data
(63) Continuation-in-part of application No. 14/628,563, filed on Feb. 23, 2015.
(60) Provisional application No. 62/081,162, filed on Nov. 18, 2014.

(51) Int. Cl. A61K 9/16; A61K 31/55; A61K 9/19; A61K 31/197; A61K 31/4166; A61K 31/53; A61K 9/00; A61K 31/195

(52) U.S. Cl. CPC A61K 9/1647; A61K 9/0085; A61K 31/55; A61K 31/195; A61K 31/197; A61K 31/4166; A61K 31/53; A61K 9/19

(57) ABSTRACT

Provided herein are methods for treating epilepsy or seizure disorders comprising administering a composition to a subject, the composition comprising an anticonvulsant agent and a biodegradable carrier, wherein the agent is incorporated within the biodegradable carrier.

### CROSS-REFERENCE TO RELATED APPLICATIONS

The present application is a continuation-in-part of U.S. Ser. No. 14/628,563, filed Feb. 23, 2015, which claims priority to U.S. Provisional Application No. 62/081,162, filed Nov. 18, 2014, the entire contents of both of which are incorporated herein by reference.

### TECHNICAL FIELD

Provided herein are compositions, methods, and kits for treating epilepsy or seizure disorders in a subject.

### BACKGROUND

Clinical management of epilepsy or seizure disorders comprises administration of anticonvulsants or antiepileptic drugs designed to control seizure activity in patients with either generalized, idiopathic epilepsy or symptomatic epilepsy. In cases of refractory or intractable epilepsy, where pharmacological intervention does not exert control over seizure activity, patients are treated with surgical intervention, for example, surgical resection of epileptogenic tissue. While surgical intervention represents an attractive treatment option for patients displaying pharmacoresistance, its scope is limited to cases where the epileptogenic tissue is not within close proximity to areas of eloquent cortex.

Anticonvulsants have been shown to be useful in the treatment and management of many epilepsy indications, as this class of drugs is known to exert important biochemical effects on nerve cells. Most drugs belonging to these classes, however, have short circulation half-lives and considerable side effects including, but not limited to, sedation, vertigo, diplopia, skin rash, nausea, vomiting, chronic diarrhea, aplastic anemia, thrombocytopenia, jaundice, oliguria, hypertension, cardiac dysrhythmias, chronic suppression of white blood cell counts, and hyponatremia. These side effects limit the potential systemic therapeutic use of anticonvulsants for the management of epilepsy. Consequently, physicians cannot always dose enough drug to have the desired antiepileptic effect without causing problematic, pleiotropic systemic side effects. In addition to undesired, systemic side effects, there exist numerous subsets of patients who either present with or develop pharmacoresistance. There exist several known hypotheses seeking to explain the mechanism inherent to these cases of pharmacoresistance, including the transporter hypothesis, which describes inadequate transport of anticonvulsant agents across the blood-brain barrier that results in insufficient concentrations of the agents at the site of epileptogenic tissue. Local delivery of anticonvulsants designed specifically to bypass the blood-brain barrier would abrogate these pleiotropic, systemic side effects and enable their therapeutic intervention for the management of epilepsy. For example, a localized injection of a depot formulation of an anticonvulsant agent would permit the use of a lower initial dose than would be required for systemic or oral administration of the agent because the depot would establish therapeutically efficacious concentrations of the agent specifically at the desired site of action.

There remains an outstanding need for formulations comprising, consisting of, or consisting essentially of anticonvulsant agents that possess physical characteristics that are consistent with clinical translation as an injectable and that can bypass the blood-brain barrier to provide desirable release profiles locally to epileptogenic tissue.

### SUMMARY

Provided herein are methods for treating epilepsy or seizure disorders in a subject comprising administering to a subject having epilepsy or seizure disorders a composition comprising, consisting of, or consisting essentially of an anticonvulsant agent and a biodegradable carrier are also disclosed herein.

Further provided are kits for producing compositions for treating epilepsy or seizure disorders in a subject.

### BRIEF DESCRIPTION OF THE DRAWINGS

FIG. 1 illustrates an exemplary biodegradable, polymeric nanoparticle or microparticle releasing an anticonvulsant agent.

FIG. 2 shows a representative scanning electron micrograph (SEM) of poly(D,L-lactide-co-glycolide) (PLGA) microparticles incorporating the anticonvulsant carbamazepine fabricated by solvent extraction/evaporation, single oil-in-water emulsification.

FIG. 3 shows a representative particle size distribution for of PLGA microparticles incorporating the anticonvulsant carbamazepine fabricated by solvent extraction/evaporation, single oil-in-water emulsification, as measured by aqueous solution phase laser diffraction (d[50]=17.5 microns and d[3,2]=10.1 microns, median and mean hydrodynamic diameters, respectively).

FIG. 4 shows examples of sustained, controlled release kinetic profiles of the anticonvulsant carbamazepine from PLGA microparticles comprising 50:50 PLGA with inherent viscosities of (a) 0.15-0.25 dL/g and (b) 0.55-0.75 dL/g.

FIG. 5 shows examples of sustained, controlled release kinetic profiles of the anticonvulsant carbamazepine from PLGA microparticles comprising 65:35 PLGA with inherent viscosities of (a) 0.55-0.75 dL/g.

FIG. 6 shows an example of a sustained, controlled release kinetic profile of the anticonvulsant carbamazepine from poly(D,L-lactide) (PLA) microparticles comprising PLA with a molecular weight of 75-120 kDa.

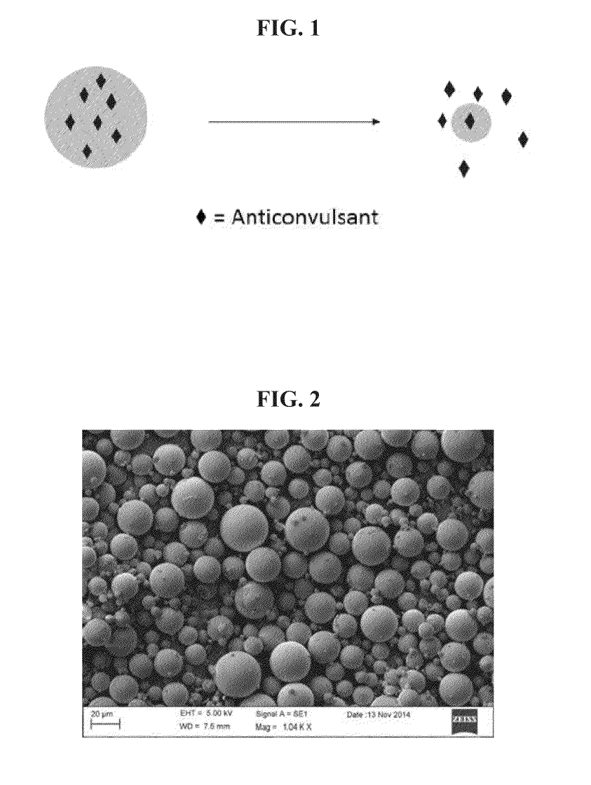

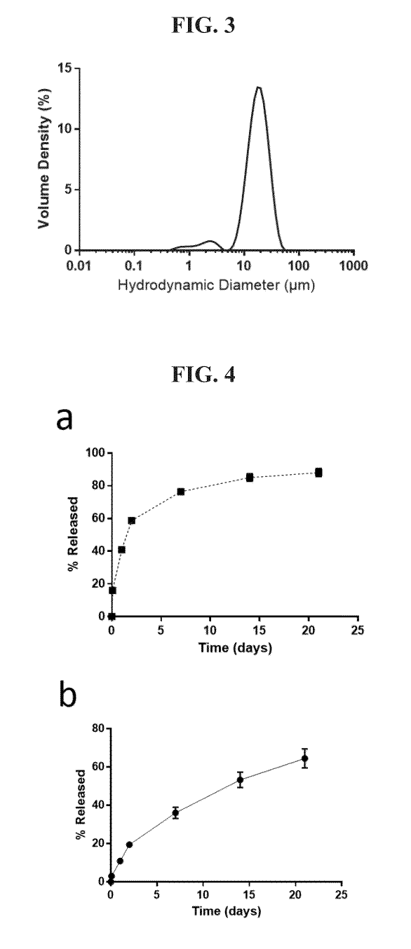

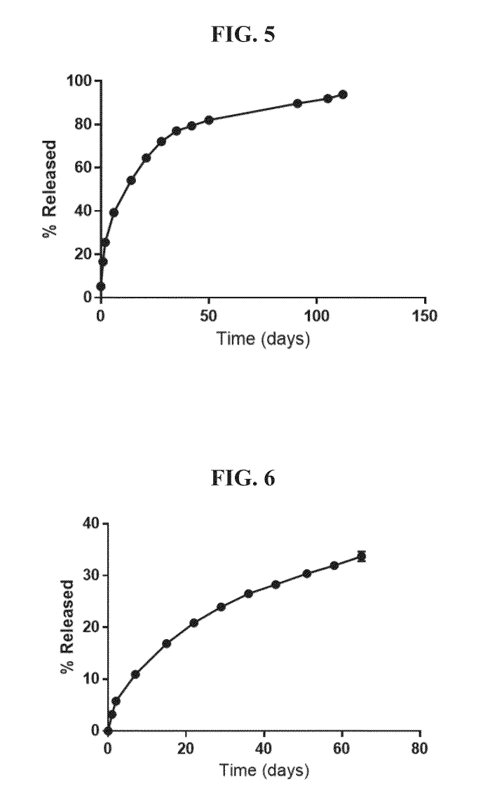

### DETAILED DESCRIPTION OF ILLUSTRATIVE EMBODIMENTS

The disclosed compositions, methods, and kits may be understood more readily by reference to the following detailed description taken in connection with the accompanying figures, which form a part of this disclosure. It is to be understood that the disclosed compositions, methods, and kits are not limited to the specific compositions, methods, and kits described and/or shown herein, and that the terminology used herein is for the purpose of describing particular embodiments by way of example only and is not intended to be limiting of the claimed compositions, methods, and kits. Also, as used in the specification including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Further, reference to values stated in ranges include each and every value within that range. All ranges are inclusive and combinable. Similarly, when values are expressed as approximations, by use of the antecedent “about,” it will be understood that the particular value forms another embodiment.

It is to be appreciated that certain features of the disclosed compositions, methods, and kits which are, for clarity, described herein in the context of separate embodiments, may also be provided in combination in a single embodiment. Conversely, various features of the disclosed compositions, methods, and kits that are, for brevity, described in the context of a single embodiment, may also be provided separately or in any subcombination.

The term “about” when used in reference to numerical ranges, cutoffs, or specific values is used to indicate that the recited values may vary by up to as much as 25% from the listed value. As many of the numerical values used herein are experimentally determined, it should be understood by those skilled in the art that such determinations can, and often times will, vary among different experiments. The values used herein should not be considered unduly limiting by virtue of this inherent variation. The term “about” is used to encompass variations of ±25% or less, variations of ±20% or less, variations of 10% or less, variations of ±5% or less, variations of ±1% or less, variations of ±0.5% or less, or variations of ±0.1% or less from the specified value.

As used herein, “administering to said subject” and similar terms indicate a procedure by which the described anticonvulsant agents or compositions, together or separately, are introduced into, implanted in, injected into, or applied onto a subject such that target cells, tissues, or segments of the body of the subject are contacted with the agent.

The terms “near” and “around” when used in reference to the site of administration of the described anticonvulsant agents or compositions should be understood by those skilled in the art to mean administered to the anatomical area of interest within the limits of traditionally practiced surgical and image-guided surgical procedures. For example, administration “near” the relevant anatomical site refers to a location that is not directly within or on the site, but sufficiently close to the site to provide a therapeutically relevant effect thereon. Those of ordinary skill in the art can readily determine the maximum distance from a given anatomical site that will be sufficient to provide a therapeutically relevant effect using a composition according to the present disclosure having a known concentration of active ingredient.

For purposes of the present disclosure, a substance is “biodegradable” if it is capable of being at least partially broken down within and cleared by the human body over time by natural biological, biochemical, and/or physiological processes. For example, carriers comprising polyesters, such as, poly(lactide-co-glycolides) (PLGA), poly(lactides) (PLA), or copolymers of PLGA or PLA with poly(ethylene glycol) (PEG), which are broken down by the human body by hydrolytic and enzymatic cleavage, through interaction with water and esterases, respectively, are thus referred to as biodegradable carriers.

“Pharmaceutically acceptable” refers to those properties and substances which are acceptable to the patient from a pharmacological/toxicological point of view and to the manufacturing pharmaceutical chemist from a physical/chemical point of view regarding composition, formulation, stability, patient acceptance, and bioavailability.

“Pharmaceutically acceptable carrier” refers to a medium that does not interfere with the effectiveness of the biological activity of the active ingredient(s) and is not toxic to the host to which it is administered.

“Therapeutically effective dose” refers to an amount of a composition, as described herein, effective to achieve a particular biological or therapeutic result such as, but not limited to, biological or therapeutic results disclosed, described, or exemplified herein. The therapeutically effective dose may vary according to factors such as the disease state, age, sex, and weight of the individual, and the ability of the composition to cause a desired response in a subject. Such results may include, but are not limited to, the treatment of epilepsy or seizure disorders, as determined by any means suitable in the art.

The terms “treating” or “treatment” refer to any success or indicia of success in the attenuation or amelioration of an injury, pathology or condition, including any objective or subjective parameter such as abatement, remission, diminishing of symptoms or making the injury, pathology, or condition more tolerable to the patient, slowing in the rate of inflammation, making the final point of inflammation less debilitating, improving a subject's physical or mental well-being, or prolonging the length of survival. The treatment may be assessed by objective or subjective parameters; including the results of a physical examination, neurological examination, or psychiatric evaluations.

As used herein, “exposed on the surface” means that at least a portion of the anticonvulsant agent is not covered or encased by the biodegradable carrier and is accessible from the exterior of the biodegradable carrier. The anticonvulsant agent exposed on the surface can be fully exposed, such that the entire agent is on the surface of the biodegradable carrier, or can be partially exposed, such that only a portion of the agent is on the surface of the biodegradable carrier. The anticonvulsant agent that is exposed on the surface of the biodegradable carrier can be bound to the surface of the biodegradable carrier through, for example, covalent or non-covalent bonds, or can be incorporated within the biodegradable carrier such that a portion of the agent is exposed on the surface.

As used herein, “incorporated within” means that the anticonvulsant agent is at least partially covered by, contained within, encased in, or entrapped by the biodegradable carrier. In such circumstances, the anticonvulsant agent may or may not be exposed on the surface of the biodegradable carrier. Depending on the type of biodegradable carrier present in the composition, the anticonvulsant agent may be located in a void space, such as a core, of the biodegradable carrier or dispersed within the biodegradable carrier with the potential for being exposed on the surface, or any combination thereof. In some embodiments, the anticonvulsant agent can be dispersed or distributed within the biodegradable carrier, and not partially exposed on the surface of the biodegradable carrier. In other embodiments, the anticonvulsant agent can be partially exposed on the surface of the biodegradable carrier. In other embodiments, the anticonvulsant agent can be both dispersed or distributed within the biodegradable carrier and partially exposed on the surface of the biodegradable carrier. In yet other embodiments, the anticonvulsant agent can be located in a void space of the biodegradable carrier. In yet other embodiments, the anticonvulsant agent can be both located in a void space of the biodegradable carrier and exposed on the surface of the biodegradable carrier.

Biodegradable, polymeric microparticles and nanoparticles represent an attractive means to achieve the desired local delivery of therapeutic agents, often by administration of a depot formulation. These particles can be fabricated by a variety of techniques to incorporate neurologically active therapeutic agents, including, anticonvulsants. The fabrication technique dictates the physical, chemical, and mechanical properties of the resulting particles. Thus, to achieve desired therapeutically efficacious concentrations and durations, the fabrication technique and polymer must be selected appropriately. For example, buoyant poly(D,L-lactide-co-glycolide) (PLGA) microspheres were created by oil-in-oil emulsification to encapsulate and deliver hydrophilic small-molecule agents (e.g., inosine) for intrathecal administration for the treatment of central nervous system disorders (WO 2004/047768).

The formulation of anticonvulsants within different biodegradable implants or carriers has been examined for achieving sustained therapeutic efficacy in epilepsy [see, e.g., Halliday et al., Adv Drug Deliv Rev., 2012, 64(10):953-64]. To these ends, the anticonvulsant, carbamazepine, has been studied as a model drug for incorporation within these types of devices [Klose et al., Inter J Pharmaceutics., 2011, 404:75-82; Barakat et al., Drug Deliv., 2006, 13(1):9-18; Pepic et al., J Microencapsulation., 2013, 30(2):151-160]; however, in these reports, the fabrication technique, polymer, and size were not selected appropriately to yield a clinically relevant drug delivery system for the treatment of epilepsy or seizure disorders. For example, different water-in-oil-in-water (w/o/w) double emulsification techniques have been utilized to fabricate either large, highly porous [Klose et al., Inter J Pharmaceutics., 2011, 404:75-82] or large, solid matrix [Barakat et al., Drug Deliv., 2006, 13(1):9-18] biodegradable poly(D,L-lactide-co-glycolide) (PLGA) microparticles incorporating carbamazepine. In both reports, the large particle size is necessary to extend the release duration, however, these forms are impractical for clinical translation as an injectable. Additionally, the intentional high, micro-scale porosity of the microparticles fabricated by Klose et al., limits the ability for sustained, long-term release applications. Further, the carbamazepine within the solid matrix microparticles fabricated by Barakat et al., is predominantly distributed on or within close proximity to the microparticle surface. This predominantly surface-associated carbamazepine gives rise to the observed high initial burst release and inability to sustain the release of therapeutically relevant concentrations. Furthermore, Pepic et al., utilized an oil-in-water (o/w) single emulsification technique to fabricate large, porous poly(E-caprolactone) (PCL) microspheres incorporating carbamazepine. In this report, the high hydrophobic character and crystallinity of the PCL causes extremely slow biodegradation that is too slow to be clinically relevant as a biodegradable carrier for therapeutic delivery in the treatment of epilepsy or seizure disorders. This slow biodegradation combined with the porosity inherent to the PCL polymer matrix causes the carbamazepine release mechanism to be solely diffusion-based, rather than biodegradation-based, necessitating large particle sizes to extend release. These large particle sizes reported are also impractical for clinical translation as an injectable. Further, the carbamazepine within the polymer matrix was observed to be predominantly distributed on or within close proximity to the microparticle surface. This predominantly surface-associated carbamazepine gives rise to the observed high initial burst release and inability to sustain the release of therapeutically relevant concentrations.

The present disclosure provides methods for utilizing compositions that are formulated specifically to enable 1) control of anticonvulsant agent incorporation, including substantially even distribution throughout the polymer matrix, 2) control over anticonvulsant agent release rate, 3) clinically relevant biodegradation rates, and 4) control over the duration of anticonvulsant agent release at therapeutically efficacious concentrations, including sustained efficacious release for an extended period of time, such as one day or more, from nanoparticles, microparticles, or any combination thereof, for the treatment of epilepsy or seizure disorders.

Further, the present disclosure provides methods for utilizing compositions that are formulated specifically to enable control over hydrodynamic diameter. The hydrodynamic diameter of the biodegradable carrier represents an important characteristic which influences 1) anticonvulsant incorporation, 2) anticonvulsant release rate, 3) biodegradation and clearance rate, 4) administration site residence duration, and 5) the ability to enable clinical administration of the composition as an injectable without necessitating a change to the standard of care.

Disclosed herein are methods for treating epilepsy or seizure disorders in a subject. In some embodiments, the methods comprise utilizing compositions comprising an anticonvulsant agent and a biodegradable carrier. In some embodiments, the methods comprising utilizing compositions consisting of an anticonvulsant agent and a biodegradable carrier. In yet other embodiments, the methods comprise utilizing compositions consisting essentially of an anticonvulsant agent and a biodegradable carrier.

Suitable biodegradable carriers include, but are not limited to, a nanoparticle, a microparticle, or any combination thereof. In some embodiments, the biodegradable carrier is a nanoparticle. In some embodiments, the biodegradable carrier is a microparticle. In some embodiments, the biodegradable carrier is a nanoparticle. In some embodiments, the biodegradable carrier is a nanoparticle.

Suitable classes of nanoparticles or microparticles include, but are not limited to, polymeric. Further, said nanoparticles or microparticles may be solid, hollow, or a mixture thereof. Further, said nanoparticles or microparticles may be porous, wherein the porosity is defined solely by the density and packing arrangement of the polymer matrix and the incorporated anticonvulsant agent.

Polymeric nanoparticles can have a mean hydrodynamic diameter up to 1 micron, as measured by dynamic light scattering in aqueous solution, wherein the hydrodynamic diameter is derived solely from the fabrication process in the absence of sieving the lyophilized product. Suitable instrumentation for aqueous solution phase dynamic light scattering includes the Malvern Instruments™ ZetaSizer® Nano ZS, wherein the mean is derived from the intensity distribution obtained with cumulants analysis. Polymeric microparticles can have a median and/or mean hydrodynamic diameter greater than or equal to 1 micron and up to about 25 microns, inclusive, as measured by laser diffraction in aqueous solution, wherein the hydrodynamic diameter is derived solely from the fabrication process in the absence of sieving the lyophilized product. Suitable instrumentation for aqueous solution phase laser diffraction includes the Malvern Instruments™ Mastersizer® 3000 equipped with the Hydro MV unit, where median and mean hydrodynamic diameter are calculated as d[50] and d[3,2], respectively. For example, microparticles can be fabricated via solvent extraction/evaporation, single oil-in-water emulsification to have a median hydrodynamic diameter (d[50]) of 18 microns, as measured by laser diffraction in aqueous solution, by precisely controlling the shear-rate and viscosity of the emulsion. Further, the disclosed compositions have sufficiently small median and/or mean hydrodynamic diameters up to 25 microns, inclusive, to enable clinical administration as an injectable without changing the standard of care.

Suitable anticonvulsant agents include, but are not limited to, carbamazepine, pregabalin, phenytoin, gabapentin, topiramate, oxcarbazepine, clobazam, clonazepam, lamotrigine, nitrazepam, retigabine, rufinamide, or tiagabine. In some embodiments, the anticonvulsant agent is carbamazepine. In some embodiments, the anticonvulsant agent is phenytoin. In some embodiments, the anticonvulsant agent is gabapentin. In some embodiments, the anticonvulsant agent is pregabalin. In other embodiments, the anticonvulsant agent is topiramate. In yet other embodiments, the anticonvulsant agent is lamotrigine.

The disclosed compositions can comprise, consist of, or consist essentially of an anticonvulsant agent and a biodegradable carrier. In some embodiments, the composition comprises, consists of, or consists essentially of carbamazepine and a nanoparticle. In some embodiments, the composition comprises, consists of, or consists essentially of carbamazepine and a microparticle. In some embodiments, the composition comprises, consists of, or consists essentially of phenytoin and a nanoparticle. In some embodiments, the composition comprises, consists of, or consists essentially of phenytoin and a microparticle. In some embodiments, the composition comprises, consists of, or consists essentially of gabapentin and a nanoparticle. In some embodiments, the composition comprises, consists of, or consists essentially of gabapentin and a microparticle. In some embodiments, the composition comprises, consists of, or consists essentially of pregabalin and a nanoparticle. In some embodiments, the composition comprises, consists of, or consists essentially of pregabalin and a microparticle. In some embodiments, the composition comprises, consists of, or consists essentially of topiramate and a nanoparticle. In some embodiments, the composition comprises, consists of, or consists essentially of topiramate and a microparticle. In some embodiments, the composition comprises, consists of, or consists essentially of oxcarbazepine and a nanoparticle. In some embodiments, the composition comprises, consists of, or consists essentially of oxcarbazepine and a microparticle. In some embodiments, the composition comprises, consists of, or consists essentially of lamotrigine and a nanoparticle. In some embodiments, the composition comprises, consists of, or consists essentially of lamotrigine and a microparticle.

Anticonvulsant agents also include mixtures of carbamazepine, pregabalin, phenytoin, gabapentin, topiramate, lamotrigine and/or oxcarbazepine within the same biodegradable carrier. For example, and without intent to be limiting, in some aspects the composition can comprise carbamazepine and pregabalin within a microparticle.

In some embodiments, the anticonvulsant agent can be formulated to comprise up to 1% by weight, inclusive, of the biodegradable carrier. In some embodiments, the anticonvulsant agent can be formulated to comprise up to 5% by weight, inclusive, of the biodegradable carrier. In some embodiments, the anticonvulsant agent can be formulated to comprise up to 10% by weight, inclusive, of the biodegradable carrier. In some embodiments, the anticonvulsant agent can be formulated to comprise up to 15% by weight, inclusive, of the biodegradable carrier. In some embodiments, the anticonvulsant agent can be formulated to comprise up to 20% by weight, inclusive, of the biodegradable carrier. In some embodiments, the anticonvulsant agent can be formulated to comprise up to 25% by weight, inclusive, of the biodegradable carrier. In some embodiments, the anticonvulsant agent can be formulated to comprise up to 50% by weight, inclusive, of the biodegradable carrier.

Throughout the present disclosure, the phrase “the anticonvulsant agent” can refer to more than one anticonvulsant agent if more than one such agent is present in the composition. For example, when only one anticonvulsant agent is contained within the biodegradable carrier, a reference to release of “60% of the anticonvulsant agent” means that there is release of 60% of the sole present anticonvulsant. When more than one anticonvulsant agent is contained within the biodegradable carrier, language referring to release of “60% of the anticonvulsant agent”, means that 60% of the total complement of anticonvulsant agents is released. Thus, if the composition includes 3 mg of a first anticonvulsant agent and 3 mg of a second anticonvulsant agent, then release of “60% of the anticonvulsant agent” can mean that 60% of the total complement of 6 mg of anticonvulsant agents is released.

Biodegradable carriers can comprise, consist of, or consist essentially of a number of materials suitable for delivering an anticonvulsant agent to a subject, including synthetically derived, biodegradable polymers. Exemplary polymers include, but are not limited to, poly(lactides) (PLA), poly(glycolides) (PGA), poly(lactide-co-glycolides) (PLGA), or copolymers of said polymers with poly(ethylene glycol)(PEG), or any combination thereof. In some embodiments, the biodegradable carrier comprises, consists of, or consists essentially of a synthetically derived biodegradable polymer. Additionally, in some embodiments, the synthetically derived biodegradable polymer can be poly(lactic-co-glycolic acid) (PLGA), having a lactic acid and glycolic acid content ranging from 0-100% for each monomer. For example, in some aspects, the biodegradable polymer can be a 50:50 PLGA, where 50:50 refers to the ratio of lactic to glycolic acid. In some embodiments, the biodegradable carrier comprises, consists of, or consists essentially of a copolymer. For example, in some embodiments, the biodegradable polymer can be a copolymer of poly(ethylene glycol) (PEG) and poly(lactic-co-glycolic acid) (PLGA), having a lactic acid and glycolic acid content ranging from 0-100% for each monomer.

Biodegradable carriers can be configured to be injected into a subject. For example, in some aspects, the biodegradable carrier comprises a nanoparticle that is configured to be injected into a subject. In other aspects, the biodegradable carrier comprises a microparticle that is configured to be injected into a subject. For injection into a subject, the microparticle must have a median and/or mean hydrodynamic diameter of not more than 25 microns, inclusive, as measured by the aforementioned aqueous solution phase laser diffraction instrumentation.

Biodegradable carriers can also be configured to be implanted into a subject. Implants can be any size and shape suitable for delivering an anticonvulsant to or near the site of epileptogenic tissue.

Biodegradable carriers can further comprise one or more surface modifications. Examples of suitable surface modification include, but are not limited to, functional group modifications, PEGylation or affinity-based targeting moieties. In some embodiments, the biodegradable carrier can be PEGylated. Surface modifications can prevent the carrier from migrating from the site of administration, abrogate the foreign body response, and/or minimize clearance by immune system cells.

The anticonvulsant agent can be exposed on the surface of the biodegradable carrier, incorporated within the biodegradable carrier, or both. In some embodiments, the anticonvulsant agent is incorporated within the biodegradable carrier.

When the anticonvulsant agent is incorporated within the biodegradable carrier, the process of incorporation may be accomplished using solvent extraction/evaporation, oil-in-water (o/w) single emulsification in the presence of a stabilizing surfactant. Suitable surfactants for stabilizing this oil-in-water emulsion include, but are not limited to, poly(vinyl alcohol) (PVA), polysorbate 80, polysorbate 85, poly(ethylene glycol), or any combination thereof.

When the anticonvulsant agent is incorporated within the biodegreadable carrier, exemplary polymers for forming the biodegradable carrier include, but are not limited to, PLGA, PLA, PLGA-PEG and PLA-PEG block copolymers, or any combination thereof.

The biodegradable carrier for use in an incorporated system can be chosen to begin to degrade within any suitable time frame following preparation for administration of the composition to a subject. In some embodiments, the biodegradable carrier can begin to degrade upon resuspension in aqueous media. In some embodiments, the biodegradable carrier can begin to degrade upon administration of the composition to a subject.

Degradation, diffusion, or any combination thereof, can lead to the controlled release of the anticonvulsant agent from the biodegradable carrier. In some embodiments, the biodegradable carrier releases less than 60% of the anticonvulsant agent over about 3 hours. In some embodiments, the biodegradable carrier releases less than 60% of the anticonvulsant agent over about 6 hours. In some embodiments, the biodegradable carrier releases less than 60% of the anticonvulsant agent over about 12 hours. In some embodiments, the biodegradable carrier releases less than 60% of the anticonvulsant agent over about 1 day. In some embodiments, the biodegradable carrier releases less than 60% of the anticonvulsant agent over about 2 days. In some embodiments, the biodegradable carrier releases less than 60% of the anticonvulsant agent over about 3 days. In some embodiments, the biodegradable carrier releases less than 60% of the anticonvulsant agent over about 4 days. In some embodiments, the biodegradable carrier releases less than 60% of the anticonvulsant agent over about 5 days. In some embodiments, the biodegradable carrier releases less than 60% of the anticonvulsant agent over about 6 days. In some embodiments, the biodegradable carrier releases less than 60% of the anticonvulsant agent over about 7 days. In some embodiments, the biodegradable carrier releases less than 60% of the anticonvulsant agent over about 8 days. In some embodiments, the biodegradable carrier releases less than 60% of the anticonvulsant agent over about 9 days. In some embodiments, the biodegradable carrier releases less than 60% of the anticonvulsant agent over about 10 days. In some embodiments, the biodegradable carrier releases less than 60% of the anticonvulsant agent over about 12 days. In some embodiments, the biodegradable carrier releases less than 60% of the anticonvulsant agent over about 14 days. In some embodiments, the biodegradable carrier releases less than 60% of the anticonvulsant agent over about 18 days. In some embodiments, the biodegradable carrier releases less than 60% of the anticonvulsant agent over about 21 days. In some embodiments, the biodegradable carrier releases less than 60% of the anticonvulsant agent over about 28 days. In some embodiments, the biodegradable carrier releases less than 60% of the anticonvulsant agent over about 35 days. In some embodiments, the biodegradable carrier releases less than 60% of the anticonvulsant agent over about 42 days. In some embodiments, the biodegradable carrier releases less than 60% of the anticonvulsant agent over about 56 days. In some embodiments, the biodegradable carrier releases less than 60% of the anticonvulsant agent over about 3 months. In some embodiments, the biodegradable carrier releases less than 60% of the anticonvulsant agent over about 4 months. In some embodiments, the biodegradable carrier releases less than 60% of the anticonvulsant agent over about 5 months. In some embodiments, the biodegradable carrier releases less than 60% of the anticonvulsant agent over about 6 months. In some embodiments, the biodegradable carrier releases less than 60% of the anticonvulsant agent over about 7 months. In some embodiments, the biodegradable carrier releases less than 60% of the anticonvulsant agent over about 8 months. In some embodiments, the biodegradable carrier releases less than 60% of the anticonvulsant agent over about 9 months. In some embodiments, the biodegradable carrier releases less than 60% of the anticonvulsant agent over about 10 months. In some embodiments, the biodegradable carrier releases less than 60% of the anticonvulsant agent over about 12 months.

Degradation of the biodegradable carrier can lead to the controlled release of and/or delivery of the anticonvulsant agent, thus providing a therapeutically effective dose of the agent to the subject. In some embodiments, the biodegradable carrier provides a therapeutically effective dose of the agent for up to 3 hours. In some embodiments, the biodegradable carrier provides a therapeutically effective dose of the agent for up to 6 hours. In some embodiments, the biodegradable carrier provides a therapeutically effective dose of the agent for up to 12 hours. In some embodiments, the biodegradable carrier provides a therapeutically effective dose of the agent for up to 1 day. In some embodiments, the biodegradable carrier provides a therapeutically effective dose of the agent for up to 2 days. In some embodiments, the biodegradable carrier provides a therapeutically effective dose of the agent for up to 3 days. In some embodiments, the biodegradable carrier provides a therapeutically effective dose of the agent for up to 4 days. In some embodiments, the biodegradable carrier provides a therapeutically effective dose of the agent for up to 5 days. In some embodiments, the biodegradable carrier provides a therapeutically effective dose of the agent for up to 6 days. In some embodiments, the biodegradable carrier provides a therapeutically effective dose of the agent for up to 7 days. In some embodiments, the biodegradable carrier provides a therapeutically effective dose of the agent for up to 8 days. In some embodiments, the biodegradable carrier provides a therapeutically effective dose of the agent for up to 9 days. In some embodiments, the biodegradable carrier provides a therapeutically effective dose of the agent for up to 10 days. In some embodiments, the biodegradable carrier provides a therapeutically effective dose of the agent for up to 12 days. In some embodiments, the biodegradable carrier provides a therapeutically effect dose of the agent for up to 14 days. In some embodiments, the biodegradable carrier provides a therapeutically effect dose of the agent for up to 18 days. In some embodiments, the biodegradable carrier provides a therapeutically effective dose of the agent for up to 3 weeks. In some embodiments, the biodegradable carrier provides a therapeutically effective dose of the agent for up to 1 month. In some embodiments, the biodegradable carrier provides a therapeutically effective dose of the agent for up to 2 months. In some embodiments, the biodegradable carrier provides a therapeutically effective dose of the agent for up to 3 months. In some embodiments, the biodegradable carrier provides a therapeutically effective dose of the agent for up to 4 months. In some embodiments, the biodegradable carrier provides a therapeutically effective dose of the agent for up to 5 months. In some embodiments, the biodegradable carrier provides a therapeutically effective dose of the agent for up to 6 months. In some embodiments, the biodegradable carrier provides a therapeutically effective dose of the agent for up to 7 months. In some embodiments, the biodegradable carrier provides a therapeutically effective dose of the agent for up to 8 months. In some embodiments, the biodegradable carrier provides a therapeutically effective dose of the agent for up to 9 months. In some embodiments, the biodegradable carrier provides a therapeutically effective dose of the agent for up to 10 months. In some embodiments, the biodegradable carrier provides a therapeutically effective dose of the agent for up to 12 months.

Degradation of the biodegradable carrier can lead to the controlled release of and/or delivery of the anticonvulsant agent, providing a therapeutically effective dose of the agent to the subject, while maintaining systemic blood plasma concentrations of the anticonvulsant agent that are lower than those associated with oral dosing or administration. In some embodiments, the blood plasma concentration of the anticonvulsant agent can be 1/1000 or less than the blood plasma concentration associated with oral dosing or administration. In some embodiments, the blood plasma concentration of the anticonvulsant agent can be 1/500 or less than the blood plasma concentration associated with oral dosing or administration. In some embodiments, the blood plasma concentration of the anticonvulsant agent can be 1/100 or less than the blood plasma concentration associated with oral dosing or administration. In other embodiments, the blood plasma concentration of the anticonvulsant agent can be below detection limits of analytical measurements.

Pharmaceutical agents may also be included in the compositions described herein. In some aspects, the pharmaceutical agents may stabilize the composition, allow it to be readily administered to a subject, increase its ability to treat epilepsy or seizure disorders, or otherwise make the composition suitable for therapeutic use in a subject. Accordingly, the described composition may further comprise a pharmaceutically acceptable carrier or excipient, as would be known to an individual skilled in the relevant art. In view of the inclusion of pharmaceutical agents in some of the described compositions, disclosed herein are also pharmaceutical compositions having an anticonvulsant and a biodegradable carrier, as provided herein. The described pharmaceutical compositions for delivery or injection of the described compositions may be administered to a subject in order to maintain the ability to treat epilepsy or seizure disorders in the subject over a prolonged period of time. For example, composition viscosity and concentration of the agent may be altered to increase the half-life of composition's active ingredients.

The disclosed compositions for use in the present methods may be formulated as any of various preparations that are known and suitable in the art, including those described and exemplified herein. In some embodiments, the pharmaceutical compositions are aqueous formulations. Aqueous solutions may be prepared by admixing the described compositions in water or suitable physiologic buffer, and optionally adding suitable colorants, preservatives, stabilizing and thickening agents, ions such as calcium or magnesium, and the like as desired. Aqueous suspensions may also be made by dispersing the described compositions in water or physiologic buffer with viscous material, such as natural or synthetic gums, resins, methylcellulose, sodium carboxymethylcellulose, and other well-known suspending agents.

When the disclosed compositions for use in the present methods are prepared as aqueous suspensions, the suspensions may be formulated by dispersing the present biodegradable carrier and active agent within injectable, in situ cross-linking hydrogel solution precursors, including, but not limited to, naturally derived polymers (e.g. polysaccharides) and/or synthetically derived polymers (e.g. PEG, PGA-PEG-PGA, PLA-PEG-PLA, PLGA-PEG-PLGA). The resulting compositions may then be administered to a subject, for example, by injection. Accordingly, a hydrogel may function as an excipient in which the biodegradable carrier and active agent are dispersed.

The disclosed compositions for use in the present methods may also be prepared as liquid formulations and solid form preparations which are intended to be converted, shortly before use, to liquid preparations. Such liquids include solutions, suspensions, syrups, slurries, and emulsions. Liquid preparations may be prepared by conventional means with pharmaceutically acceptable additives such as suspending agents (e.g., sorbitol syrup, cellulose derivatives or hydrogenated edible fats or oils); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). These preparations may contain, in addition to the active agent, stabilizers, buffers, dispersants, thickeners, solubilizing agents, and the like. The compositions may be in powder or lyophilized form for constitution with a suitable vehicle such as sterile water, physiological buffer, saline solution, or alcohol, before use. The compositions may be formulated for injection into a subject. For injection, the compositions described may be formulated in aqueous solutions such as water or alcohol, or in physiologically compatible buffers such as Hanks's solution, Ringer's solution, or physiological saline buffer. The solution may contain one or more formulatory agents such as suspending, stabilizing or dispersing agents. Injection formulations may also be prepared as solid form preparations which are intended to be converted, shortly before use, to liquid form preparations suitable for injection, for example, by constitution with a suitable vehicle, such as sterile water, saline solution, or alcohol before use.

The disclosed compositions for use in the present methods can be administered by injection or implantation. For example, the composition can be injected or surgically placed onto or near the focal lesion interest. Additionally, the composition can be injected or surgically placed into a resection cavity. Local delivery allows a therapeutic concentration of the composition to be delivered to the nerve in question, without the systemic levels, including, but not limited to, blood plasma concentrations, rising as high as when oral or systemic delivery is used for the same effect. Consequently, the systemic side effects can be greatly reduced or entirely eliminated.

The disclosed compositions for use in the present methods can be injected by a number of routes, including, but not limited to, intracranially, epidurally, intravenously, intra-arterially, transdermally, subcutaneously, intra-articularly, intramuscularly, perineurally, percutaneously, or any combination thereof. Alternatively, the disclosed compositions for use in the present methods can be implanted at or near identified sites implicated in the origination or cause of epilepsy or seizure disorders.

In some embodiments, the composition can be administered near or onto a neuron. For example, in some aspects, the composition can be injected near or onto a neuron. In other aspects, the composition can be surgically implanted near or onto a neuron. In other embodiments, the composition can be administered near or onto a synapse. In some aspects, the composition can be injected near or onto a synapse. In other aspects, the composition can be surgically implanted near or onto a synapse. In other embodiments, the composition can be administered near or onto a focal lesion. In some aspects, the composition can be injected near or onto a focal lesion. In other aspects, the composition can be surgically implanted near or onto a focal lesion. In other embodiments, the composition can be administered into or around a resection cavity. In some aspects, the composition can be injected into or around a resection cavity. In other aspects, the composition can be surgically implanted into or around a resection cavity. In other embodiments, the composition can be administered into or around neocortex. In some aspects, the composition can be injected into or around neocortex. In other aspects, the composition can be surgically implanted into or around neocortex. In other embodiments, the composition can be administered into or around the temporal lobe. In some aspects, the composition can be injected into or around the temporal lobe. In other aspects, the composition can be surgically implanted into or around the temporal lobe. In other embodiments, the composition can be administered into or around the frontal lobe. In some aspects, the composition can be injected into or around the frontal lobe. In other aspects, the composition can be surgically implanted into or around the frontal lobe. In other embodiments, the composition can be administered into or around the parietal lobe. In some aspects, the composition can be injected into or around the parietal lobe. In other aspects, the composition can be surgically implanted into or around the parietal lobe. In other embodiments, the composition can be administered into or around the occipital lobe. In some aspects, the composition can be injected into or around the occipital lobe. In other aspects, the composition can be surgically implanted into or around the occipital lobe. In other embodiments, the composition can be administered into or around the hippocampus. In some aspects, the composition can be injected into or around the hippocampus. In other aspects, the composition can be surgically implanted into or around the hippocampus.

The disclosed methods can be used to treat epilepsy or seizure disorders caused by a number of ailments, diseases, disorders, and/or injuries, including, but not limited to, epilepsy or seizure disorders caused by trauma, traumatic brain injury, fever, infection, meningitis, encephalitis, stroke, vascular abnormalities or malformations, blood sugar disturbance, electrolyte disturbance, intracranial hemorrhage, structural abnormalities or malformations of the brain, brain tumor or cancer, Alzheimer's disease, genetic disorders, tuberous sclerosis, or any combination thereof. Further, the disclosed methods can be used to treat idiopathic epilepsy or seizure disorders.

The disclosed methods for utilizing the described compositions for treating epilepsy include, for example, administering the composition onto or near the focal lesion implicated in causing the epileptic seizures.

The disclosed methods for utilizing the described compositions for treating epilepsy include, for example, administering the composition into or around a resection cavity.

The disclosed methods for utilizing the described compositions for treating epilepsy include, for example, administering the composition into or around the temporal lobe.

The disclosed methods for utilizing the described compositions for treating epilepsy include, for example, administering the composition into or around the frontal lobe.

The disclosed methods for utilizing the described compositions for treating epilepsy include, for example, administering the composition into or around the parietal lobe.

The disclosed methods for utilizing the described compositions for treating epilepsy include, for example, administering the composition into or around the occipital lobe.

Also provided herein are kits for producing the disclosed compositions for use in the present methods to treat epilepsy or seizure disorders in a subject; the kit comprising, consisting of, or consisting essentially of an anticonvulsant agent, a biodegradable carrier, and instructions for producing the composition. The instructions may describe the steps and reagents for producing the composition by solvent extraction/evaporation, oil-in-water single emulsification, by spray drying, or by precipitation using a solvent/non-solvent system. Such steps and reagents may be in accordance with those that the present application discloses for solvent extraction/evaporation, oil-in-water single emulsification, spray drying, and precipitation using a solvent/non-solvent system.

### Examples

### Microencapsulated Anticonvulsant Agent by Solvent Extraction/Evaporation, Single Oil-In-Water Emulsification

Biodegradable, polymeric microparticles were fabricated using a solvent extraction/evaporation, single oil-in-water (o/w) emulsification method. PLGA (0-20 wt %) and carbamazepine (0-20 wt %) were dissolved in a suitable, volatile organic solvent (e.g. dichloromethane, ethyl acetate). The resulting polymer solution dispersant phase was added to an aqueous continuous phase containing 1-5% (w/v) of surfactant (PVA) under constant shear rate mixing to create a single o/w microemulsion. The resulting stable microemulsion was subsequently added to an evaporation bath containing 100 mL of deionized water containing a trace concentration (0-0.5% (w/v)) of surfactant (PVA) under stirring at 350 rpm for 3 hours to effectively extract and evaporate the organic solvent. The hardened microparticles were then collected, purified with deionized water, and lyophilized.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed:

1. A method of treating a subject having epilepsy or seizure disorders comprising administering to said subject a composition comprising:
an anticonvulsant agent; and
a carrier comprising poly(lactide-co-glycolides), poly(lactides), or a copolymer of said poly(lactide-co-glycolides) or said poly(lactides) with poly(ethylene glycol), or any combination thereof,
wherein the anticonvulsant agent is incorporated within the carrier by solvent extraction/evaporation, oil-in-water single emulsification, by spray drying, or by precipitation using a solvent/non-solvent system.

2. The method of claim 1, wherein the composition is administered intracranially, as to bypass the blood-brain barrier, in said subject.

3. The method of claim 1, wherein the composition is administered near or around a focal lesion of said subject.

4. The method of claim 1, wherein the composition is administered into or around a resection cavity of said subject.

5. The method of claim 1, wherein the composition is administered into or around the temporal lobe of said subject.

6. The method of claim 1, wherein the composition is administered into or around the frontal lobe of said subject.

7. The method of claim 1, wherein the composition is administered into or around the parietal lobe of said subject.

8. The method of claim 1, wherein the composition is administered into or around occipital lobe of said subject.

9. The method of claim 1, wherein the composition is injected or surgically implanted in said subject.

10. The method of claim 1, wherein the epilepsy or seizure disorder is caused by epilepsy or seizure disorders caused by trauma, traumatic brain injury, fever, infection, meningitis, encephalitis, stroke, vascular abnormalities or malformations, blood sugar disturbance, electrolyte disturbance, intracranial hemorrhage, structural abnormalities or malformations of the brain, brain tumor or cancer, Alzheimer's disease, genetic disorders, tuberous sclerosis, or any combination thereof.

11. The method of claim 1, wherein the epilepsy or seizure disorder is idiopathic.

12. The method according to claim 1, wherein the anticonvulsant agent comprises carbamazepine, pregabalin, phenytoin, gabapentin, topiramate, oxcarbazepine, clobazam, clonazepam, lamotrigine, nitrazepam, retigabine, rufinamide, tiagabine, or any combination thereof.

13. The method according to claim 12, wherein the anticonvulsant agent is carbamazepine.

14. The method according to claim 12, wherein the anticonvulsant agent is gabapentin.

15. The method according to claim 12, wherein the anticonvulsant agent is pregabalin.

16. The method according to claim 12, wherein the anticonvulsant agent is phenytoin.

17. The method according to claim 12, wherein the anticonvulsant agent is lamotrigine.

18. The method according to claim 1, wherein the anticonvulsant agent is exposed on the surface of the carrier, incorporated within the carrier, or both.

19. The method according to claim 1, wherein the carrier comprises a microparticle, a nanoparticle, or any combination thereof.

20. The method according to claim 19, wherein the microparticle, nanoparticle, or any combination thereof, comprises poly(lactide), poly(lactide-co-glycolide), a copolymer of poly(lactide) and poly(ethylene glycol), or a copolymer of poly(lactide-co-glycolide) and poly(ethylene glycol), or any combination thereof.

21. The method according to claim 19, wherein the microparticle has a median hydrodynamic diameter of greater than or equal to 1 micron and up to 25 microns, inclusive, as measured by aqueous solution phase laser diffraction instrumentation.

22. The method according to claim 19, wherein the microparticle has a mean hydrodynamic diameter of greater than or equal to 1 micron and up to 25 microns, inclusive, as measured by aqueous solution phase laser diffraction instrumentation.

23. The method according to claim 19, wherein the nanoparticle has a mean hydrodynamic diameter of up to 1 micron, as measured by aqueous solution phase dynamic light scattering instrumentation.

24. The method according to claim 19, wherein the hydrodynamic diameter of the carrier is derived solely from the fabrication process in the absence of sieving the lyophilized product.

25. The method according to claim 1, wherein the carrier degrades following being administered to the subject, resulting in the release of the anticonvulsant agent.

26. The method according to claim 1, wherein the anticonvulsant agent comprises up to 50% by weight, inclusive, of the carrier.

27. The method according to claim 1, wherein the carrier releases less than 60% of the anticonvulsant agent over about 3 hours, 6 hours, 12 hours, 1 day, 2 days, 3 days, 4 days, 5 days, 6 days, 7 days, 8 days, 9 days, 10 days, 12 days, 14 days, 16 days, 18 days, 21 days, 28 days, 35 days, 42 days, 49 days, 56 days, 3 months, 4 months, 5 months, 6 months, 7 months, 8 months, 9 months, 10 months, or 12 months.

28. The method according to claim 1, wherein the carrier provides a therapeutically effective dose of the anticonvulsant agent for up to 3 hours, 6 hours, 12 hours, 1 day, 2 days, 3 days, 4 days, 5 days, 6 days, 7 days, 8 days, 9 days, 10 days, 12 days, 14 days, 18 days, 3 weeks, 1 month, 2 months, 3 months, 4 months, 5 months, 6 months, 7 months, 8 months, 9 months, 10 months, or 12 months, inclusive.

29. The method according to claim 28, wherein the carrier provides a therapeutically effective dose of the anticonvulsant agent, while maintaining systemic blood plasma concentrations of the anticonvulsant agent that are lower than those associated with oral dosing or administration.

30. The method according to claim 1, further comprising a pharmaceutically acceptable carrier or excipient.

31. A kit for use in treating a subject having epilepsy or seizure disorders comprising:
an anticonvulsant agent;
a carrier comprising poly(lactide-co-glycolides), poly(lactides), or a copolymer of said poly(lactide-co-glycolides) or said poly(lactides) with poly(ethylene glycol), or any combination thereof; and
instructions for producing said composition.

32. The kit according to claim 31 wherein said instructions are for incorporating the anticonvulsant agent within the carrier by solvent extraction/evaporation, oil-in-water single emulsification.

33. The kit according to claim 31 wherein said instructions are for incorporating the anticonvulsant agent within the carrier by spray drying.

34. The kit according to claim 31 wherein said instructions are for incorporating the anticonvulsant agent within the carrier by precipitation using a solvent/non-solvent system.
